SEQUENCE LISTING (16 sequences)

SEQ ID NO: 1 - protein, 178 aa, artificial sequence
  Oscillibacter
LAAITGKRERRRVFGRKKMEIYERRVTSMKKLLAMVLALVMTLSLAVSANAAFKDDKSISDDYAEAVAVLNGMGVFKGYEDGSFKPEGNITRAEVATIIYRIYTADVAKNDKSGLYATYNKFSDMAGAGWAQGYIGYCANAALVKGYPDGTFKPSGNVTGYEVLAMILRAVGYDKNNE

SEQ ID NO: 2 - protein, 209 aa, artificial sequence
  Oscillibacter
MKKLLAMVLALVMTLSLAVSANAAFKDDKSISDDYAESVAVLNGMGVFKGYEDGSFKPEGNITRAEVATIIYRIYTADVAKNDKSGLYATYNKFSDMAGAGWAQGYIGYCANASLVKGYPDGTFKPSGKVTGYEVLAMILRAVGYDKNNEFSGADWALHVAQTAQQLGVLDNVAKTTDLNAPASRELVAELLFQGIQKAQVTYTPAFGY

SEQ ID NO: 3 - protein, 360 aa, artificial sequence
  Oscillibacter
MKKLLAMVLALVMTLSLAVSASAVKADEKINEDYAEAVAVLNGMGVFKGYEDGSFQPKGDITRAEVSAIVYRVYTQDVKDAKASMYATYNKFSDMAGAGWAQGYIGYCANAELVKGYPDGTFKPSGKVTGYEVLAMILRAVGYDKNGEFSGADWALHVAQTAQQAGVLKNVKGVDLNAPASRELVAELLFRAVAESATVTYTPAFGYVTDKVLGNAAPTLGYKNFKLVGKDSADKWGRPATKWTYNVGDKETLVNDKPVVSYTTKVAECDIAKDLGISSAKKIEKAYIDGQQPGITSELVSTNKYGTINPLATTSYVGAQGRLTAVFYMDSDGYRIVEINTYLAKVDKVTAAKTDRNGHT

SEQ ID NO: 4 - protein, 9 aa, artificial sequence
  peptide fragment
AEVATIIYR

SEQ ID NO: 5 - protein, 8 aa, artificial sequence
  peptide fragment
IYTADVAK

SEQ ID NO: 6 - protein, 9 aa, artificial sequence
  peptide fragment
SGLYATYNK

SEQ ID NO: 7 - protein, 10 aa, artificial sequence
  peptide fragment
TTDLNAPASR

SEQ ID NO: 8 - protein, 13 aa, artificial sequence
  peptide fragment
ELVAELLFQGIQK

SEQ ID NO: 9 - protein, 10 aa, artificial sequence
  peptide fragment
GYEDGSFQPK

SEQ ID NO: 10 - protein, 10 aa, artificial sequence
  peptide fragment
GVDLNAPASR

SEQ ID NO: 11 - protein, 12 aa, artificial sequence
  peptide fragment
VLGNAAPTLGYK

SEQ ID NO: 12 - protein, 8 aa, artificial sequence
  peptide fragment
WTYNVGDK

SEQ ID NO: 13 - protein, 18 aa, artificial sequence
  peptide fragment
YGTINPLATTSYVGAQGR

SEQ ID NO: 14 - DNA, 534 bp, artificial sequence
  Oscillibacter
ttggcagcaatcacgggaaagcgggagagacggcgggtgttcggccgcaagaagatggaaatttatgaaaggagagtaacatctatgaaaaagttactcgcaatggtgctggctctggtcatgactctgtctctggccgtcagcgccaacgcagccttcaaggatgacaagagcatcagcgatgattacgctgaggccgttgccgttctgaatggcatgggtgtgttcaagggttatgaggatggttccttcaagcccgagggcaacatcacccgcgctgaggtagctacgatcatctatcgtatctacactgccgacgtcgccaagaacgataagtccggcctctatgccacttataacaagttcagcgacatggctggcgccggttgggctcagggctacatcggctactgcgccaacgccgctctcgtcaagggttatcccgacggcaccttcaagccctccggcaacgtcacgggctacgaggtcctcgccatgatcctccgcgcggttggctacgacaagaacaacgag

SEQ ID NO: 15 - DNA, 627 bp, artificial sequence
  Oscillibacter
atgaaaaagttactcgcaatggtgctggctctggtcatgactctgtctctggccgtcagcgccaacgcagccttcaaggatgacaagagcatcagcgatgattacgctgagtctgttgccgttctgaatggcatgggtgtgttcaagggttatgaggatggttccttcaagcccgagggcaacatcacccgcgctgaggtagctacgatcatctatcgtatctacactgccgacgtcgccaagaacgataagtccggcctctatgccacttataacaagttcagcgacatggctggtgccggttgggctcagggctacatcggctactgcgccaacgcctccctcgtcaagggctatcccgatggcaccttcaagccgtccggcaaggtcaccggctatgaagtcctcgccatgatcctccgcgctgtcggctacgacaagaacaacgagttctccggcgctgactgggcgctccatgttgcgcagaccgcgcagcagctcggcgttctggacaacgtggcgaagaccaccgacctgaacgctcccgcttcccgtgagctggtcgctgagctcctgttccagggcatccagaaggctcaggtcacctacaccccggccttcggctat

SEQ ID NO: 16 - DNA, 1080 bp, artificial sequence
  Oscillibacter
atgaaaaagttactcgcaatggtgctggctctggtcatgactctgtctctggccgtcagcgccagcgccgtgaaggccgacgagaagatcaacgaagattacgctgaagctgtcgccgtcctgaatggcatgggtgttttcaagggttatgaggacggttccttccagcccaagggcgacatcacccgcgctgaggtgtccgcgatcgtttatcgcgtgtacactcaggacgtcaaggatgctaaggcttccatgtacgccacctacaacaagttcagcgacatggctggcgccggttgggctcagggctacatcggctactgcgccaacgctgaactggtcaagggctatcccgacggtaccttcaagccctccggcaaggtcaccggctacgaagtcctcgctatgatcctccgcgctgtcggttacgacaagaacggtgagttctccggcgctgattgggcgctgcatgttgcgcagaccgcgcagcaggccggcgtgctcaagaacgtcaagggtgttgacctcaacgctcccgcttcccgtgagctggttgctgagctgctgttccgcgccgtcgctgagtccgccaccgtcacctacactccggccttcggctatgtgaccgataaggtgctcggcaatgctgctcccaccctcggctacaagaacttcaagctcgtcggcaaggattccgctgacaagtggggccgtcccgctaccaagtggacctacaacgtcggcgacaaggagactcttgtcaacgacaagcccgtcgtctcctacaccaccaaggttgccgagtgcgacatcgccaaggatctgggcatctcctccgctaagaagatcgagaaggcctacattgatggtcagcagcccggtattacctccgagctcgtcagcaccaacaagtacggcaccatcaacccgcttgccaccacctcctatgtcggcgctcagggccgtttgaccgcggtcttttacatggattccgacggttaccgcatcgttgagatcaacacctacctcgccaaggtcgacaaggtcaccgctgccaagaccgaccgcaacggccacacc

(19) United States
(12) Patent Application Publication
(10) Pub. No.: US 2017/0349930 A1
(43) Pub. Date: Dec. 7, 2017

(54) DIAGNOSTIC MARKERS FOR CROHN'S DISEASE

(71) Applicants: INSTITUT NATIONAL DE LA RECHERCHE AGRONOMIQUE, PARIS (FR); CENTRE NATIONAL DE LA RECHERCHE SCIENTIFIQUE, PARIS (FR); UNIVERSITE DE STRASBOURG, STRASBOURG (FR)

(72) Inventors: CATHERINE JUSTE, LE PERRAY EN YVELINES (FR); JOEL DORE, VITRY SUR SEINE (FR); ALAIN GUILLOT, FORGES LES BAINS (FR); CHRISTINE CARAPITO, SCHILTIGHEIM (FR); ALVARO SEBASTIAN VACA JACOME, STRASBOURG (FR)

(73) Assignees: Institut National De La Recherche Agronomique, Paris (FR); Centre National De La Recherche Scientifique, Paris (FR); Universite De Strasbourg, Strasbourg (FR)

(21) Appl. No.: 15/536,750

(22) PCT Filed: Dec. 18, 2015
(86) PCT No.: PCT/FR2015/053612

§ 371 Date: Jun. 16, 2017

(30) Foreign Application Priority Data
Dec. 19, 2014 (FR) 1462867

(51) Int. Cl. C12Q 1/04; G01N 33/68

(52) U.S. Cl. CPC C12Q 1/04; G01N 33/6893; G01N 2800/52; G01N 2800/065

(57) ABSTRACT

The invention relates to markers for Crohn's disease and to specific compounds for detecting said markers. The invention also relates to methods for detecting, diagnosing and monitoring the progression of Crohn's disease as well as to methods for evaluating the efficacy of a treatment for said disease, and to compositions and kits that can be used for implementing said methods.

The invention relates to the field of medicine and more particularly to the diagnosis of Crohn's disease. It identifies, for the first time, specific markers for Crohn's disease within inflammatory bowel diseases (IBDs) as well as specific compounds for detecting said markers. The invention also relates to methods for detecting, diagnosing and monitoring the progression of Crohn's disease and to methods for evaluating the efficacy of a treatment for said disease, and to diagnostic compositions and kits that can be used for implementing said methods.

### TECHNOLOGICAL BACKGROUND OF THE INVENTION

Chronic inflammatory bowel diseases (or IBD) include Crohn's disease and ulcerative colitis, two diseases characterized by inflammation of the wall of a portion of the digestive tract, linked to hyperactivity of the digestive immune system.

Crohn's disease is an IBD that can affect any segment of the digestive tract, from the mouth to the anus. This pathology can affect the intestine, and more particularly the terminal ileum (ileitis) and the ascending colon (colitis), and may be accompanied by extraintestinal (articular, cutaneous, ocular, etc.) manifestations. All the layers of the intestinal wall, from the mucous membrane to the serous membrane, may then be affected. Crohn's disease may appear at any age, including in young children, with however the highest incidence in the second or third decade of life, and a tendency towards a greater number of female patients. Crohn's disease will progress throughout one's lifetime with inflammatory flare-ups of which the frequency, the intensity and the duration are unpredictable and highly variable from one patient to another. Crohn's disease affects quality of life, calls for personalized treatments that are inconvenient and costly but unfortunately never curative and often associated with adverse effects that contribute to the decline in quality of life. Periods of hospitalization may be frequent and nearly 80% of patients will one day require surgery. Only less than 5% of these patients will be free of endoscopic lesions 10 years after the operation (Peyrin-Biroulet et al., 2010). According to recent epidemiological meta-analyses, the relative risk of colorectal and small intestine cancers and the risk of mortality, adjusted for age, are significantly higher in subjects with Crohn's disease. The cost to the health system for treating patients with Crohn's disease in Europe is estimated at 3 billion euros, excluding biological treatments and indirect costs (Juillerat et al., 2010).

The etiology of Crohn's disease remains obscure and there are no recognized rules of prevention, apart from smoking cessation (Johnson et al., 2005). However, studies implicate genetic susceptibility (Mathew et al., 2008; Cho, 2008; Cleynen et al., 2013), environmental factors (Carbonnel et al., 2009), and alterations of the intestinal microbiota (Seksik et al., 2003; Mangin et al., 2004; Manichanh et al., 2006; Kleessen et al., 2002; Scanlan et al., 2006 and Gophna et al., 2006) associated with inappropriate activation of the intestinal mucosal immune system (Laroux et al., 2001; Artis, 2008; Landers et al., 2002).

Today, diagnosis of Crohn's disease is very difficult because there is no specific symptom of the disease but rather manifestations in common with other ailments, such as ulcerative colitis in particular, but also gastrointestinal tract ailments, simple gastroenteritis, irritable bowel syndrome or infectious colitis. All these intestinal disorders, which affect 10% to 20% of the population, are the subject of a very high number of office visits, and consequently represent a real challenge for the practitioner in terms of identifying their causes. However, there is a genuine need to identify Crohn's disease patients specifically in order to provide them as soon as possible with a suitable therapeutic treatment that could enable them to avoid the often-necessary surgical procedure.

To date, and in the absence of possible distinction, a pragmatic approach is adopted with firstly an antibiotic treatment, and secondly the introduction of major treatments (immunosuppressants, biotherapy) and the implementation of invasive complementary examinations. Absent the ability to cure, the treatments (salicylate derivatives, systemic or topical corticoids, immunosuppressants and, more recently, anti-TNF-α biological treatments) serve to make the inflammatory flare-ups less frequent and to lower the intensity of the symptoms experienced. But these immunosuppressive and biological treatments are not innocuous, the more so as they are generally taken for prolonged periods by young patients. The use of thiopurines (immunosuppressants) is notably associated with a clear increase of the risk of nonmelanoma skin cancer and of lymphomas (Beaugerie et al., 2009; Peyrin-Biroulet et al., 2011; Sokol et al., 2009). The long-term risks of biological treatments are still poorly understood. Providing an effective, notably early diagnostic test would allow patients to be treated at the beginning of the disease, thus limiting the risks of severe complications and irreversible lesions. Thus, when Crohn's disease is detected in a child or an adolescent, the initial treatment can be simply nutritional. An example of a dietary food for medical use employed in this context is Modulen® (complete drinkable feed notably containing TGF-beta, an immune response regulator). Certain patients respond very well to the treatments established very early in life and thus may be brought into remission for years.

There is currently no robust and specific marker for Crohn's disease that makes it possible to diagnose this pathology or to monitor its progression. Diagnosing Crohn's disease is based today on a body of arguments both clinical (digestive and general signs and other manifestations) and paraclinical (biology, endoscopic examinations and others), with some patients having to wait years before a name is given to their disease. Thus, the average period of time between the first manifestations and the first office visit is 5 months, with a diagnosis provided after an average of 2.6 years.

However, the early diagnosis of Crohn's disease assumes a major importance since, by acting sufficiently early, it is possible to slow the development of potentially irreversible lesions (Colombel et al., 2010). Conversely, it would be highly desirable to be able to exclude in a reliable and rapid manner, typically as of the first office visit, the existence of Crohn's disease so as to avoid extensive, costly, inconvenient and useless examinations and treatments often associated with adverse effects. Moreover, there is a genuine need to monitor patients in order to evaluate the improvement or the worsening of the disease and consequently to adjust the treatments as precisely as possible.

The inventors satisfy this need thanks to the present invention and to the markers identified in the present text which make it possible to diagnose Crohn's disease specifically, typically to distinguish said disease from ulcerative colitis, in a subject without the need for extensive or invasive clinical and paraclinical examinations.

### SUMMARY OF THE INVENTION

The inventors describe markers for diagnosing, for the first time in a reliable manner, Crohn's disease, as well as tools for detecting said markers. The inventors were able to show the absence or the presence in an abnormally low quantity of these markers in the digestive system of patients with Crohn's disease in a period of remission or outside inflammatory flare-ups. Among the markers of interest identified, preferred markers, of bacterial origin, correspond to the amino acid sequences SEQ ID NO: 1, SEQ ID NO: 2, SEQ ID NO: 3 and fragments of said amino acid sequences, and to the nucleic acid sequences encoding said amino acid sequences and fragments of said sequences.

Described in the present text are thus a compound binding all or a portion of a bacterium expressing a peptide sequence comprising an amino acid sequence selected from SEQ ID NO: 1, SEQ ID NO: 2, SEQ ID NO: 3 and a fragment thereof, or comprising a nucleic acid encoding said peptide sequence, and a kit, in particular a diagnostic kit, comprising such a compound and at least one means for revealing the bond between said compound and all or a portion of the bacterium, as well as the use thereof to test for possible Crohn's disease in a subject or to monitor the progression of Crohn's disease in a subject treated for said Crohn's disease.

Also described are a diagnostic composition comprising at least one compound as described in the present text and a pharmaceutically acceptable support, and a kit, in particular a diagnostic kit, comprising such a composition and at least one means for revealing the bond between said compound and all or a portion of the bacterium, as well as the use thereof to test for possible Crohn's disease in a subject or to monitor the progression of Crohn's disease in a subject treated for said Crohn's disease.

The present text also describes the use of a diagnostic kit for preparing a diagnostic composition according to the invention or for obtaining information useful in the diagnosis of Crohn's disease.

Another object of the invention relates to a method for detecting the possible presence in a biological sample from a subject to be tested for Crohn's disease of at least one bacterium expressing a peptide sequence comprising an amino acid sequence selected from SEQ ID NO: 1, SEQ ID NO: 2, SEQ ID NO: 3 and a fragment thereof, or comprising a nucleic acid encoding said peptide sequence, or a portion of said bacterium.

A method for diagnosing Crohn's disease comprising implementing, in vitro or ex vivo, a detection method according to the invention on a biological sample from a subject to be tested is also described. It is characterized in that the absence of the bacterium or a portion thereof, or the detection of the bacteria or portions thereof, in a quantity below a control value within the sample, makes it possible to diagnose Crohn's disease in said subject, the presence within the sample of the bacteria or portions thereof in a quantity above said control value making it possible, conversely, to exclude the existence of Crohn's disease in said subject.

Another method described in the present text makes it possible to evaluate, in a subject, the therapeutic efficacy of a treatment for Crohn's disease. This method comprises implementing, in vitro or ex vivo, a detection method according to the invention on a biological sample from a subject treated for Crohn's disease, the appearance of or an increase in the quantity of bacteria or portions thereof revealing the efficacy of said treatment, and the disappearance of or the reduction in the quantity of bacteria or portions thereof revealing, conversely, the inefficacy of said treatment, when compared with a control value, for example when compared with the quantity of bacteria or portions thereof detected in said subject before said subject was treated for Crohn's disease.

### DETAILED DESCRIPTION OF THE INVENTION

The present invention relates to biological markers for Crohn's disease, in particular markers of bacterial origin, enabling the relevant diagnosis of said disease in a subject, the monitoring of the progression of said disease and the confirmation of the efficacy of a treatment for same. The invention also relates to methods for detecting said markers and tools for implementing said methods, typically compounds (preferably detectable compounds) binding said markers.

The inventors have in particular revealed the presence in significant quantities, in the digestive system of subjects not suffering from Crohn's disease (also identified as “healthy subjects” in the present text), markers, in particular markers of bacterial origin, and have shown their presence in very small quantities, even in undetectable quantities, in subjects with Crohn's disease. The invention thus describes, for the first time and in a particularly advantageous manner, methods for diagnosing Crohn's disease, for monitoring the progression and/or the efficacy of a treatment for Crohn's disease, based on measuring in a biological sample from a subject to be tested at least one marker as described in the present text. The marker of interest is preferably a bacterium expressing a peptide sequence comprising an amino acid sequence selected from SEQ ID NO: 1, SEQ ID NO: 2, SEQ ID NO: 3 and a fragment thereof (i.e., a fragment of one of said SEQ ID NO: 1, SEQ ID NO: 2 or SEQ ID NO: 3) or comprising a nucleic acid encoding said peptide sequence or said fragment thereof. The marker of interest typically corresponds to a portion of said bacterium, which may be for example an amino acid sequence or a nucleic acid sequence as described in the present text. The present description also provides tools for implementing said methods. The absence of detection or the detection in a quantity below a control value within a biological sample makes it possible to diagnose with certainty Crohn's disease in a subject, while the presence within the sample in a quantity above a control value makes it possible, conversely, to exclude the existence of this very specific disease even if the tested subject indeed suffers from an intestinal ailment, in particular from an IBD, typically from ulcerative colitis.

The expression “Crohn's disease” typically refers to the persistent inflammation of the walls and the deep layers of the digestive tract of a subject. This inflammation may be characterized by local thickening of the intestinal walls and by fissures and by wounds. It is often associated with intestinal disorders, diarrhea, fever or intense fatigue. In some cases, nondigestive symptoms may appear in the joints, skin and/or eyes. It is characterized chiefly by attacks of stomach pain and diarrhea, which may last several weeks or several months. Fatigue, weight loss and even undernutrition may then occur. The disease progresses with flare-ups interrupted by periods of remission which may last several months. Sometimes the symptoms of a recurrence (or of an attack) are so intense (inability to eat, hemorrhage, diarrhea, etc.) that hospitalization becomes necessary.

In the context of the present invention, the “early stage” of Crohn's disease is typically associated with the symptoms classically found in both chronic digestive disorders and chronic inflammatory bowel diseases, which include notably abdominal pain, diarrhea, rectal bleeding, weight loss, fatigue and fever.

By “subject” is meant an animal, typically a human or non-human mammal, preferably a human being. Particular subject populations correspond to subjects predisposed to the development of Crohn's disease (which may, for example, result from a family history of chronic inflammatory bowel disease (IBD) or even precisely Crohn's disease) or to subjects suspected of having Crohn's disease for any other reason. Another particular subject population corresponds to subjects not having any symptom or known predisposition.

A subject preferentially concerned by the present invention has chronic digestive problems or an unclassified (i.e., not specifically identified) chronic inflammatory bowel disease.

The present invention stems from the discovery and the characterization by the inventors of biological markers or “biomarkers” characteristic of Crohn's disease. Said markers make it possible to diagnose or, conversely, to exclude with certainty the existence of Crohn's disease in a subject and have the advantage of being able to be assayed from non-invasive or slightly invasive samples, typically from a sample comprising fecal matter and/or fecal water, as explained below.

The invention thus describes effective biological markers, in particular nucleic acids, proteins, polypeptides, peptides or combinations of peptides, typically of bacterial origin, that can be used to diagnose Crohn's disease as early as possible.

Any one of the markers according to the invention and any combination thereof, the detection of which in a subject makes it possible to determine, even at an early stage, the existence of Crohn's disease, can also be used to monitor the progression of said disease or the efficacy of a treatment for same. Each marker according to the invention advantageously makes it possible further to distinguish Crohn's disease from other IBDs, and in particular from ulcerative colitis.

In the present invention, the term “bacterium” is understood in the broad sense and corresponds typically to the bacterium having preserved its integrity or to a mixture of bacteria having preserved their integrity. A preferred bacterium in the context of the invention expresses a peptide sequence comprising an amino acid sequence selected from SEQ ID NO: 1,

SEQ ID NO: 2, SEQ ID NO: 3 and a fragment thereof (i.e., a fragment of one of said SEQ ID NO: 1, SEQ ID NO: 2 or SEQ ID NO: 3) or comprises a nucleic acid encoding said peptide sequence or said fragment thereof.

By “portion of a bacterium” is meant any molecule or amino acid sequence or nucleic acid sequence (nucleotide sequence) from a bacterium of interest, for example from the bacterium Alkaliphilus oremlandii, preferably from a bacterium of the genus Oscillibacter, or from several bacteria of interest, and able to be recognized by a compound of the invention. These “portion(s) of a bacterium” correspond in the present application to “markers” or “biomarkers” as defined above and may also be referred to as “targets”.

In a particular embodiment, the portion of the bacterium used as a marker corresponds to a molecule or an amino acid sequence, typically selected from a protein, a polypeptide, a peptide or a combination of peptides (for example a peptide complex), whether natural or artificial. This molecule may correspond to a fragment of capsule, of cell wall, of plasma membrane, of flagellum or of endospore or to a component of the cytoplasm characteristic of one or more bacteria of interest. The marker corresponds for example to an extracellular structure, or to a polysaccharide portion of the external wall. The markers comprising amino acids from the bacterium/bacteria of interest are preferably peptides, polypeptides or proteins found on the extracellular portion of a bacterium, typically “exposed” on the bacterial wall. Preferably, these bacteria are able to be detected easily with or without alteration of the bacterial wall.

A preferred polypeptide marker comprises, or consists of, a sequence selected from SEQ ID NO: 1, SEQ ID NO: 2, SEQ ID NO: 3, more preferably SEQ ID NO: 2 or SEQ ID NO: 3, and a fragment of one or the other of said sequences SEQ ID NO: 1, SEQ ID NO: 2 or SEQ ID NO: 3.

SEQ ID NO: 1
>2136630|2107226|GL0047428_O2_[Lack_3'-
end]_[mRNA]_locus=scaffold20302_4:1307:1840:+|NA|
K01448,K02035|map00550
LAAITGKRERRRVFGRKKMEIYERRVTSMKKLLAMVLALVMTLSLAVSANA
AFKDDK|SISDDYAEAVAVLNGMGVFK|GYEDGSFKPEGNITR|
AEVATIIYR|IYTADVAK|NDK|SGLYATYNK|FSDMAGAGWAQGYIGYCA
NAALVKGYPDGTFKPSGN|VTGYEVLAMILR|AVGYDKNNE
SEQ ID NO: 2
>2361546|2299029|GL0014029_V1_[Lack_3'-
end]_[mRNA]_locus=C4048270_1:32:658:+|NOG09437|
K01448,K02035,K08654|map00550
MKKLLAMVLALVMTLSLAVSANAAFKDDK|SISDDYAESV
AVLNGMGVFK|GYEDGSFKPEGNITR|AEVATIIYR|IYTADVAK|
NDK|SGLYATYNK|FSDMAGAGWAQGYIGYCANASLVK|
GYPDGTFKPSGK|VTGYEVLAMILR|AVGYDK|NNEFSGADWALHVAQ
TAQQLGVLDNVAK|TTDLNAPASR|ELVAELLFQGIQK|AQVTYTPAFGY
SEQ ID NO: 3
>2769615|2671322|GL0089028_V1_[Lack_3'-
end]_[mRNA]_locus=scaffold19302_2:2:1081:-|NA|
K01448|map00550
MKKLLAMVLALVMTLSLAVSASAVKADEKINEDYAEAVAVLNGMGVFK|
GYEDGSFQPK|GDITR|AEVSAIVYR|VYTQDVKDAKASMYATYNKFSD
MAGAGWAQGYIGYCANAELVK|GYPDGTFKPSGK|VTGYEVLAMILR|
AVGYDK|NGEFSGADWALHVAQTAQQAGVLK|NVK|GVDLNAPASR|ELV
AELLFRAVAESATVTYTPAFGYVTDK|VLGNAAPTLGYK|NFKLVGKDSA
DKWGRPATK|WTYNVGDK|ETLVNDKPVVSYTTKVAECDIAKDLGISSAK
KIEKAYIDGQQPGITSELVSTNK|YGTINPLATTSYVGAQGR|LTAVFYM
DSDGYRIVEINTYLAKVDKVTAAKTDRNGHT

Examples of peptide markers of interest correspond to markers comprising, or consisting of, a sequence selected from SEQ ID NO: 4 (AEVATIIYR), SEQ ID NO: 5 (IYTADVAK), SEQ ID NO: 6 (SGLYATYNK), SEQ ID NO: 7 (TTDLNAPASR), SEQ ID NO: 8 (ELVAELLFQGIQK), SEQ ID NO: 9 (GYEDGSFQPK), SEQ ID NO: 10 (GVDLNAPASR), SEQ ID NO: 11 (VLGNAAPTLGYK), SEQ ID NO: 12 (WTYNVGDK) and SEQ ID NO: 13 (YGTINPLATTSYVGAQGR), or a combination of several of said peptides.

A preferred peptide marker comprises, or consists of, a sequence selected from SEQ ID NO: 8 (ELVAELLFQGIQK), SEQ ID NO: 11 (VLGNAAPTLGYK) and SEQ ID NO: 13 (YGTINPLATTSYVGAQGR), or a combination of several of said peptides.

In a particular embodiment, the combination of target peptides used comprises or consists of a sequence selected from SEQ ID NO: 8, SEQ ID NO: 11, SEQ ID NO: 13 or a combination thereof.

In another particular embodiment, the portion of the bacterium/bacteria of interest, typically a bacterium of the genus Oscillibacter, used as a marker is a nucleic acid molecule in the form of a DNA molecule, an RNA molecule, a plasmid, etc., from, or obtained from, the bacterium/bacteria of interest. The nucleic acid sequence is for example a gene from a bacterium of interest. The nucleic acid sequences used comprise, or consist of, an mRNA sequence selected preferably from the sequences SEQ ID NOs: 14, 15 or 16. The nucleic acids having the sequences SEQ ID NOs: 14-16 were in particular identified by the inventors from biological samples from healthy subjects or from subjects with Crohn's disease, by genome analysis techniques (as explained in the experimental section of the present description).

>2136630|2107226|GL0047428_O2_[Lack_3′-
end]_[mRNA]_locus=scaffold20302_4:1307:1840:
+|NA|K01448,K02035|map00550
SEQ ID NO: 14
TTGGCAGCAATCACGGGAAAGCGGGAGAGACGGCGGGTGTTCGGCCGCA
AGAAGATGGAAATTTATGAAAGGAGAGTAACATCTATGAAAAAGTTACT
CGCAATGGTGCTGGCTCTGGTCATGACTCTGTCTCTGGCCGTCAGCGCC
AACGCAGCCTTCAAGGATGACAAGAGCATCAGCGATGATTACGCTGAGG
CCGTTGCCGTTCTGAATGGCATGGGTGTGTTCAAGGGTTATGAGGATGG
TTCCTTCAAGCCCGAGGGCAACATCACCCGCGCTGAGGTAGCTACGATC
ATCTATCGTATCTACACTGCCGACGTCGCCAAGAACGATAAGTCCGGCC
TCTATGCCACTTATAACAAGTTCAGCGACATGGCTGGCGCCGGTTGGGC
TCAGGGCTACATCGGCTACTGCGCCAACGCCGCTCTCGTCAAGGGTTAT
CCCGACGGCACCTTCAAGCCCTCCGGCAACGTCACGGGCTACGAGGTCC
TCGCCATGATCCTCCGCGCGGTTGGCTACGACAAGAACAACGAG
>2361546|2299029|GL0014029_V1_[Lack_3′-
end]_[mRNA]_locus=C4048270_1:32:658:
+|NOG09437|K01448,K02035,K08654|map00550
SEQ ID NO: 15
ATGAAAAAGTTACTCGCAATGGTGCTGGCTCTGGTCATGACTCTGTCTC
TGGCCGTCAGCGCCAACGCAGCCTTCAAGGATGACAAGAGCATCAGCGA
TGATTACGCTGAGTCTGTTGCCGTTCTGAATGGCATGGGTGTGTTCAAG
GGTTATGAGGATGGTTCCTTCAAGCCCGAGGGCAACATCACCCGCGCTG
AGGTAGCTACGATCATCTATCGTATCTACACTGCCGACGTCGCCAAGAA
CGATAAGTCCGGCCTCTATGCCACTTATAACAAGTTCAGCGACATGGCT
GGTGCCGGTTGGGCTCAGGGCTACATCGGCTACTGCGCCAACGCCTCCC
TCGTCAAGGGCTATCCCGATGGCACCTTCAAGCCGTCCGGCAAGGTCAC
CGGCTATGAAGTCCTCGCCATGATCCTCCGCGCTGTCGGCTACGACAAG
AACAACGAGTTCTCCGGCGCTGACTGGGCGCTCCATGTTGCGCAGACCG
CGCAGCAGCTCGGCGTTCTGGACAACGTGGCGAAGACCACCGACCTGAA
CGCTCCCGCTTCCCGTGAGCTGGTCGCTGAGCTCCTGTTCCAGGGCATC
CAGAAGGCTCAGGTCACCTACACCCCGGCCTTCGGCTAT
2769615|2671322|GL0089028_V1_[Lack_3′-
end]_[mRNA]_locus=scaffold19302_2:2:1081:
-|NA|K01448|map00550
SEQ ID NO: 16
ATGAAAAAGTTACTCGCAATGGTGCTGGCTCTGGTCATGACTCTGTCTC
TGGCCGTCAGCGCCAGCGCCGTGAAGGCCGACGAGAAGATCAACGAAGA
TTACGCTGAAGCTGTCGCCGTCCTGAATGGCATGGGTGTTTTCAAGGGT
TATGAGGACGGTTCCTTCCAGCCCAAGGGCGACATCACCCGCGCTGAGG
TGTCCGCGATCGTTTATCGCGTGTACACTCAGGACGTCAAGGATGCTAA
GGCTTCCATGTACGCCACCTACAACAAGTTCAGCGACATGGCTGGCGCC
GGTTGGGCTCAGGGCTACATCGGCTACTGCGCCAACGCTGAACTGGTCA
AGGGCTATCCCGACGGTACCTTCAAGCCCTCCGGCAAGGTCACCGGCTA
CGAAGTCCTCGCTATGATCCTCCGCGCTGTCGGTTACGACAAGAACGGT
GAGTTCTCCGGCGCTGATTGGGCGCTGCATGTTGCGCAGACCGCGCAGC
AGGCCGGCGTGCTCAAGAACGTCAAGGGTGTTGACCTCAACGCTCCCGC
TTCCCGTGAGCTGGTTGCTGAGCTGCTGTTCCGCGCCGTCGCTGAGTCC
GCCACCGTCACCTACACTCCGGCCTTCGGCTATGTGACCGATAAGGTGC
TCGGCAATGCTGCTCCCACCCTCGGCTACAAGAACTTCAAGCTCGTCGG
CAAGGATTCCGCTGACAAGTGGGGCCGTCCCGCTACCAAGTGGACCTAC
AACGTCGGCGACAAGGAGACTCTTGTCAACGACAAGCCCGTCGTCTCCT
ACACCACCAAGGTTGCCGAGTGCGACATCGCCAAGGATCTGGGCATCTC
CTCCGCTAAGAAGATCGAGAAGGCCTACATTGATGGTCAGCAGCCCGGT
ATTACCTCCGAGCTCGTCAGCACCAACAAGTACGGCACCATCAACCCGC
TTGCCACCACCTCCTATGTCGGCGCTCAGGGCCGTTTGACCGCGGTCTT
TTACATGGATTCCGACGGTTACCGCATCGTTGAGATCAACACCTACCTC
GCCAAGGTCGACAAGGTCACCGCTGCCAAGACCGACCGCAACGGCCACA
CC

Another object of the invention relates to a compound binding all or a portion of a marker of interest, typically all or a portion of a bacterium of interest, preferably of a bacterium of the genus Oscillibacter.

The expression “compound binding all or a portion of a bacterium” (more simply referred to as “compound” in the context of the present description) refers to a compound comprising, or consisting of, a molecule binding all or a portion of a bacterium, said molecule being typically selected from a molecule consisting of amino acids such as a protein, a peptide, a combination of peptides or a polypeptide; a nucleic acid such as a DNA molecule, an RNA molecule or a plasmid; an antibody; a sugar, for example an oligosaccharide or a polysaccharide; and any mixture thereof such as, for example, a peptidoglycan.

When it has a peptide nature, i.e., when it comprises a peptide, the compound may be advantageously acetylated, methylated, phosphorylated, glycosylated, or fused with another protein, peptide and polypeptide according to techniques well known to the person skilled in the art.

When it comprises, or consists of, a nucleic acid, the compound may be advantageously inserted in a DNA fragment, or a cloning vector.

In a particular embodiment, the compound consists of an antibody.

In another particular embodiment, the compound consists of a peptide construction, preferably selected from a fusion protein and a recombinant protein.

In yet another particular embodiment, the compound consists of a nucleic acid sequence which may be, for example, in the form of an aptamer.

A preferred compound according to the invention binds at least one biological marker in the context of the invention from a bacterium of interest, typically a nucleic acid molecule or a molecule comprising amino acids. Such a compound is for example selected from a molecule consisting of amino acids such as a protein, a peptide, a combination of peptides or a polypeptide; a nucleic acid such as a DNA molecule, an RNA molecule or a plasmid; an antibody; a sugar, for example an oligosaccharide or a polysaccharide; and any mixture thereof such as, for example, a peptidoglycan.

In a particular embodiment, the compound according to the invention binding at least one biological marker in the context of the invention comprises, or consists of, a construction comprising a peptide or a nucleic acid binding all or a portion of the bacterium/bacteria of interest.

The construction, when it comprises a peptide, is preferably selected from a fusion protein or a recombinant protein, typically derived from molecular engineering and obtained according to methods well known to the person skilled in the art.

A preferred compound binds, or is able to bind, a marker corresponding to an amino acid molecule from a bacterium of interest. The amino acid molecules used preferably as targets comprise, or consist of, a sequence selected from SEQ ID NO: 1, SEQ ID NO: 2 and SEQ ID NO: 3. These protein markers were identified by the inventors from biological samples from healthy subjects or subjects with Crohn's disease, by proteome analysis techniques known to the person skilled in the art.

A particularly preferred compound binds a polypeptide comprising, or consisting of, a sequence selected from SEQ ID NO: 2 and SEQ ID NO: 3.

Another preferred compound binds a peptide comprising, or consisting of, a sequence selected from SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13. A particularly preferred compound binds a peptide comprising, or consisting of, a sequence selected from SEQ ID NO: 8, SEQ ID NO: 11 and SEQ ID NO: 13.

In a particular embodiment, the compound according to the invention binding at least one biological marker in the context of the invention comprising amino acids comprises, or consists of, an antibody or an antibody fragment or derivative. Preferably, the ligand is an antibody specific for the amino acid molecule, a fragment of such an antibody (for example a Fab, a Fab′, a CDR, etc.), or a derivative of such an antibody (for example a single-chain antibody, scFv, nanobodies, etc.). The ligand is typically immobilized on a support, such as a slide, a bead, a column, a plate, etc. The presence or the quantity of the target molecule in the sample can be detected/measured by showing a complex between the target and one of its ligands. The ligand can be labeled to facilitate its detection. A second detection ligand may also be used. The well-known immunological techniques that may be used are ELISA, RIA, etc. If necessary, the quantity of detected peptide, polypeptide or protein may be compared with a control value, for example with a control value observed in subjects not having Crohn's disease.

Antibodies specific for the markers according to the invention can be produced by conventional techniques, notably by immunizing a non-human animal with an immunogen comprising the peptide, polypeptide or protein marker (or an immunogenic fragment thereof), and recovering antibodies (polyclonal) or producing cells (to produce monoclonal antibodies). Techniques for producing polyclonal or monoclonal antibodies, scFv fragments, human or humanized antibodies are described for example in Harlow et al. [Antibodies: A Laboratory Manual, CSH Press, 1988]; Ward et al. [Natural 341 (1989) 544]; Bird et al. [Science 242 (1988) 423]; WO 94/02602; U.S. Pat. No. 5,223,409; U.S. Pat. No. 5,877,293; and WO 93/01288. The immunogen can be manufactured by synthesis, or by expression, in a suitable host, of a target nucleic acid as defined above. Such a monoclonal or polyclonal antibody, and derivatives thereof having the same antigen specificity, also constitutes a compound concerned by the present invention. According to a preferred embodiment, these compounds bind, or are able to bind, a bacterium of interest, or a portion thereof (for example a gene, a protein, a polypeptide, a peptide or a combination of peptides).

In a preferred embodiment, the invention has as an object a compound as described in the present text bound to a detectable marker. This detectable marker is used for visualizing, indeed for quantifying, the marker of interest. Preferably, this detectable marker is selected from a bead, a fluorophore, a chemiluminescent fragment, a stable isotope, a particle (for example a nanoparticle) such as a magnetic particle or a metal particle that can be detected by imaging.

This detectable marker may be easily selected by the person skilled in the art as a function of the nature of the target biological marker.

The invention also relates to compositions comprising at least one compound as described in the present text, typically a compound binding at least one biological marker in the context of the invention, and uses thereof.

A diagnostic composition comprising at least one compound according to the invention and a pharmaceutically acceptable support is thus advantageously described.

A pharmaceutically acceptable support may be, for example, a substance selected from an excipient, a carrier, an adjuvant, a buffer, traditionally used with detection or diagnostic tools. The choice of such supports depends essentially on the detection or diagnostic method used.

The invention typically relates to a compound according to the invention and a diagnostic composition comprising such a compound, for use in testing for possible Crohn's disease in a subject, preferably at the earliest possible stage, from a biological sample from said subject. The invention further relates to the use of compound(s) of the invention for monitoring the progression of Crohn's disease typically in a subject treated for said disease.

In the context of the present invention, the expression “biological sample” refers to any biological sample from the subject to be tested, preferably a sample containing nucleic acids, proteins or fragments thereof. A typical sample comprises for example fluids, tissues, cells, proteins, nucleic acids. Preferably, the sample according to the invention may be obtained by any technique known per se, for example by taking a sample, by non-invasive techniques, from collections or sample banks. The sample typically corresponds to a sample for studying intestinal microbiota. Such a sample preferably comprises fecal matter (stool) and/or fecal water. The sample may also consist of a biopsy (or surgical specimen) of, or comprising, intestinal epithelial tissue or intestinal mucus. In particular embodiments, the sample is a sample of fresh matter or a sample of frozen matter.

The sample may further be pretreated to facilitate accessibility to the target molecules, for example by lysis (mechanical, chemical, enzymatic, etc.), purification, centrifugation, separation, etc. The sample may also be labeled (fluorescent, radioactive, luminescent, chemical, enzymatic labeling, etc.) so as to be detected more easily. The detectable marker is preferably selected from a bead, an inorganic particle, for example a metal particle, a fluorophore, a chemiluminescent fragment and a stable isotope. Techniques of cell lysis, of concentration or of dilution of nucleic acids or of proteins are well known to the person skilled in the art.

The invention concerns in particular the detection of genes, peptides, polypeptides or proteins derived from, and preferably characteristic of, the bacterium/bacteria of interest. Prior to the present invention, there existed no means for diagnosing Crohn's disease in a subject from a biological sample from said subject, typically from a sample of fecal matter and/or fecal water.

The invention also relates to a diagnostic kit comprising at least one compound or at least one composition according to the invention and at least one means for revealing or detecting the bond between said compound and all or a portion of a bacterium of interest.

In a particular embodiment, the kit according to the invention comprises at least one antibody specific for a marker for Crohn's disease as described in the present text and one or more detection reagents and/or buffers.

In another particular embodiment, the kit according to the invention comprises specific reagents for detecting RNA, DNA, typically cDNA, or protein or a protein fragment (for example oligonucleotide probes, primers or antibodies). In a preferred embodiment, the kit contains all the components necessary and sufficient to carry out the detection, including all controls, test protocols and, if necessary, software for analyzing and presenting the results. The kits may also comprise, for example, PCR buffers and enzymes, positive control sequences, reaction control primers, and instructions for amplifying or detecting the specific sequences.

Such kits may be used for detecting all or a portion of one or more bacteria of interest and/or for assaying said bacterium/bacteria, in a biological sample, notably for testing for possible Crohn's disease in a subject, in particular for distinguishing, within a cohort of IBD patients, subjects with Crohn's disease from those with ulcerative colitis, or for obtaining information useful in the diagnosis of Crohn's disease, or for monitoring the progression of Crohn's disease in a subject treated for said disease.

In a particular embodiment, the diagnostic kit is a kit comprising means for implementing an ELISA or a Western blot. To that end, the diagnostic kit contains a means for detecting one or more portions of the bacterium of interest, the detection means corresponding notably to one or more polyclonal or monoclonal antibodies.

The present text also relates to a kit for preparing a diagnostic composition as described in the present text.

As indicated above, the invention advantageously makes it possible to detect the presence of Crohn's disease in a subject and, once diagnosed, to monitor the progression of said disease.

A particularly advantageous object of the invention resides in a method for detecting the possible presence in a biological sample from a subject to be tested, typically from a subject to be tested for Crohn's disease, of at least one bacterium of interest or a portion thereof, for example several portions thereof (separately or in relation to one another).

The presence and/or the structure of the markers of interest may be detected/analyzed by means of techniques known to the person skilled in the art, typically using targeted quantitative proteomics technologies.

A preferred method uses mass spectrometry (MS). Selected reaction monitoring (SRM) makes it possible to quantify very precisely the marker(s) specifically detected by mass spectrometry.

In a preferred embodiment of the present invention, SRM with labeling of the peptides of interest, preferably of the peptides SEQ ID NO: 4 to SEQ ID NO: 13, is advantageously implemented. By inference, the method makes it possible to quantify very precisely the abundance of the proteins SEQ ID NO: 1 to SEQ ID NO: 3 in several different samples. These techniques, more generally referred to as targeted proteomics, are used, in the context of the present invention, to quantify in a biological sample from a subject to be tested at least one bacterium or a portion thereof (corresponding preferably to a molecule comprising amino acids, typically a peptide, a combination of peptides, a polypeptide or a protein).

Another particularly advantageous object of the invention resides in a method for detecting the possible presence in a biological sample from a subject to be tested of a marker of interest, typically at least one bacterium of interest or a portion thereof, comprising:
contacting, in vitro or ex vivo, a biological sample from the subject to be tested with a compound according to the invention binding all or a portion of the marker of interest, typically at least one bacterium of interest; and
detecting the bond between said compound and the marker of interest, typically said at least one bacterium of interest or a portion thereof, when it is present within the biological sample, and preferably measuring the quantity of said marker within the biological sample.

In a particular embodiment, the method comprises determining the presence or the quantity (relative) of one or more amino acid molecules (peptide, polypeptide or protein) as a marker from the bacterium of interest.

The detection or the assaying of such an amino acid molecule in a sample may be carried out by any technique known to the person skilled in the art. This detection is preferably carried out using a compound according to the invention capable of binding all or a portion of the bacterium of interest as described above.

In another particular embodiment, the method comprises contacting, under conditions allowing hybridization between complementary sequences, the biological sample from the subject to be tested, or nucleic acids extracted from said biological sample, and a set of specific probes for the target molecules (biomarkers) identified by the inventors for obtaining a hybridization profile, the hybridization profile being characteristic of the presence of Crohn's disease in said subject, or of the (in)efficacy of the treatment provided to the subject, as the case may be.

Various techniques for detecting a kind of nucleic acid in a biological sample can be used in the present invention, such as for example Northern Blot, selective hybridization, use of supports coated with oligonucleotide probes, nucleic acid amplification such as for example by RT-PCR, quantitative PCR or ligation-PCR, etc. Said methods may comprise the use of a nucleic probe (for example an oligonucleotide) capable of detecting selectively or specifically the target nucleic acid in the sample. Amplification may be carried out according to various methods known to the person skilled in the art, such as PCR, LCR, transcription-mediated amplification (TMA), strand displacement amplification (SDA), NASBA, the use of allele-specific oligonucleotides (ASO), allele-specific amplification, Southern blot, single-strand conformation analysis (SSCA), in situ hybridization (for example, FISH), gel migration, heteroduplex analysis, etc. If necessary, the quantity of nucleic acid detected may be compared with a control value, for example a median or mean value observed in patients not having Crohn's disease.

According to a preferred embodiment, the method comprises detecting the presence or the absence or the quantity (relative) of nucleic acid molecules.

Selective hybridization is typically carried out by using nucleic probes, preferably immobilized on a support, such as a solid or semi-solid support having at least one surface, flat or not, allowing the immobilization of nucleic probes. Such supports are for example a slide, a bead, a membrane, a filter, a column, a plate, etc. They may be made of any compatible material, notably such as glass, silica, plastic, fiber, metal, polymer, etc. The nucleic probes may be any nucleic acid (DNA, RNA, PNA, etc.), preferably single-stranded, comprising a specific sequence of a target marker as defined above. The probes may be synthetic oligonucleotides, products based on sequences of target molecules of the invention, prepared by traditional synthesis techniques. Probe pairs, one of which is a perfect match to the target molecule and the other is a mismatch, thus making it possible to estimate the background, may also be used.

The probes may be synthesized beforehand then deposited on the support, or synthesized directly in situ, on the support, according to methods known per se to the person skilled in the art. The probes may also be produced by genetic techniques, for example by amplification, recombination, ligation, etc.

Hybridization may be carried out under conventional conditions, known to and adjustable by the person skilled in the art (Sambrook, Fritsch, Maniatis (1989) Molecular Cloning, Cold Spring Harbor Laboratory Press). In particular, hybridization may be carried out under high, medium or low stringency conditions, according to the desired level of sensitivity, the quantity of material available, etc.

In a typical embodiment, the nucleic acids (or chips or supports) are prehybridized in hybridization buffer. The nucleic acids of the sample are then contacted with the probes. Preferably, the nucleic acids of the sample are labeled beforehand, by any known labeling method (radioactive, enzymatic, fluorescent, luminescent, etc.). The supports are then washed. The hybridization profile is analyzed by conventional techniques, such as for example by measuring the labeling on the support by means of a suitable instrument. The hybridization conditions may naturally be adjusted by the person skilled in the art, for example by modifying the hybridization temperature and/or the salt concentration of the buffer, as well as by adding auxiliary substances such as formamide or single-stranded DNA.

Selective amplification is preferably carried out by using a primer or a primer pair allowing amplification in the sample of all or a portion of one of the target nucleic acids, when the latter is present. The primer may be specific for a target sequence as defined above, or for a region flanking the target sequence in a nucleic acid of the sample.

A particularly advantageous object of the invention relates to a method for diagnosing Crohn's disease comprising implementing, in vitro or ex vivo, on a biological sample from a subject to be tested, a detection method as described above. The absence of the bacterium of interest or portions thereof, or the detection of the bacteria of interest or portions thereof, in a quantity below a control value within the sample, makes it possible to diagnose Crohn's disease in said subject, and the presence within the sample of the bacteria of interest or portions thereof in a quantity above said control value makes it possible, conversely, to exclude the existence of Crohn's disease in said subject.

In a particular embodiment, the diagnostic method described is used for screening, the subject exhibiting no symptom or having no known predisposition.

The invention further relates to a method for evaluating, in a subject, the therapeutic efficacy of a treatment for Crohn's disease, said method comprising implementing, in vitro or ex vivo, a detection method according to the invention on a biological sample from a subject treated for Crohn's disease, the appearance of or an increase in the quantity of the bacterium/bacteria of interest or portions thereof revealing the efficacy of said treatment, and the disappearance of or the reduction in the quantity of the bacterium/bacteria of interest or portions thereof revealing, conversely, the inefficacy of said treatment, when compared with a control value, for example when compared with the quantity of the bacteria of interest or portions thereof detected in said subject before said subject was treated for Crohn's disease.

In the context of the invention, a “control value” refers to a concentration, quantity or content of proteins, polypeptides, peptides or nucleic acids from the bacterium of interest as measured on a “reference sample” or a “control sample”, typically on a “healthy sample”, also referred to as a “normal sample” in the present text.

This control value is, for example, obtained from measurements taken from several biological samples obtained from a subject, preferably several subjects, not having Crohn's disease.

The reference sample may also correspond to a sample obtained from one or more patients with Crohn's disease. It is typically the case in the context of implementing a method for monitoring the progression of Crohn's disease or for determining the efficacy of a treatment for said disease, where the control value corresponds preferably to that determined from a sample taken from the subject to be monitored, but before the monitoring and/or the treatment of Crohn's disease in said subject has begun.

The invention also relates to a method for treating a subject identified (screened) as having Crohn's disease, using a conventional treatment for Crohn's disease which the practitioner will be able to select, preferably using a peptide or polypeptide (natural or synthetic) described in the present text or a nucleic acid sequence encoding such a peptide or polypeptide administered to the subject to be treated, for example using an expression vector.

The present invention is illustrated by the following figures and examples, which should be regarded as illustrative and nonrestrictive.

### FIGURES

FIG. 1: Identifiers, sequences and functional and phylogenetic annotation of three proteins SEQ ID NO: 1, SEQ ID NO: 2 and SEQ ID NO: 3 (mass matching against the MetaHIT-2009 databank, reannotated 2014). The peptides in bold are those seen in label-free LC-MS/MS shotgun experiments. The peptides underlined and in bold indicate the specific peptides targeted in SRM. The vertical bars delimit the peptides seen in shotgun experiments or targeted in SRM.

FIG. 2: Sequence alignment of the three proteins having the sequences SEQ ID NO: 1, SEQ ID NO: 2 and SEQ ID NO: 3 (AlignUniprot—http://www.uniprot.org/blast/)

FIG. 3: Search for the protein domains of SEQ ID NO: 1, SEQ ID NO: 2 and SEQ ID NO: 3 in the InterPro database (EMBL-EBI—http://www.ebi.ac.uk/Tools/pfa/iprscan5/)

### EXAMPLES

The inventors discovered and validated, in patients in remission or having moderate flare-ups, a panel of bacterial and human protein signals associated with Crohn's disease (Juste et al., 2014). The bacterial proteins and a few human proteins adhering to the bacterial cells are extracted from the subjects' stools. Among the myriad of proteins present, it was shown, surprisingly, that some are remarkably less abundant in the patients compared with healthy controls, matched for age, sex and tobacco use.

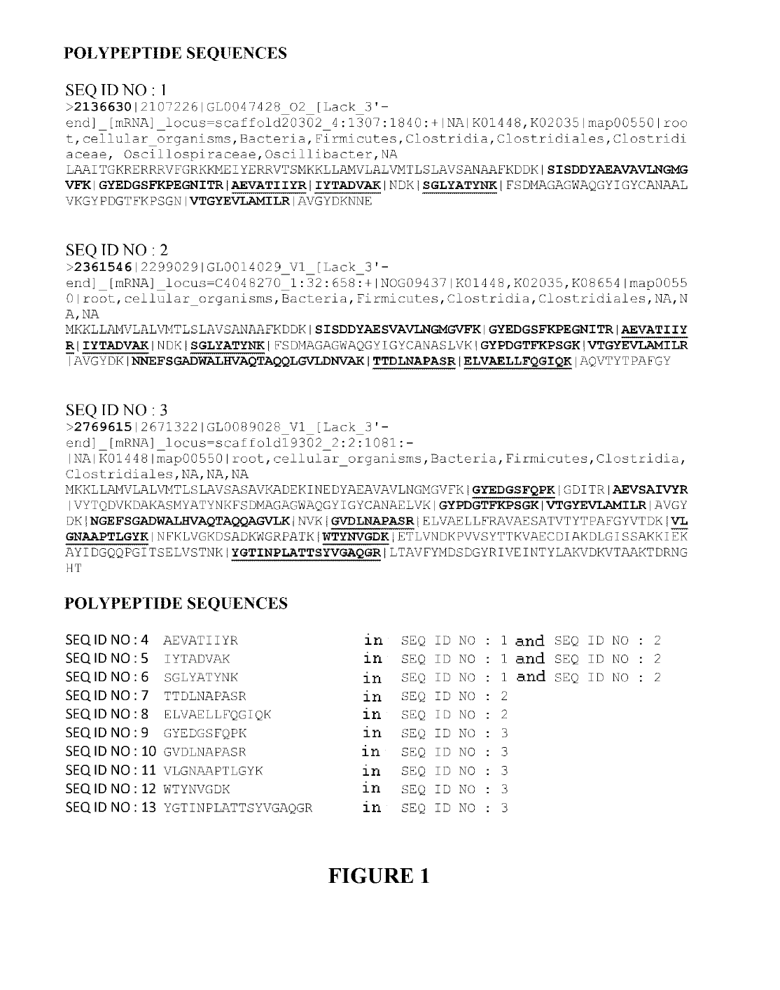

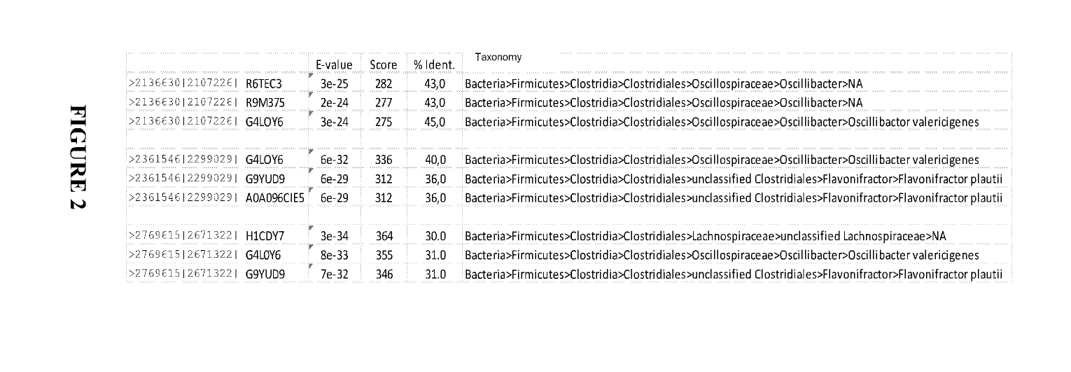

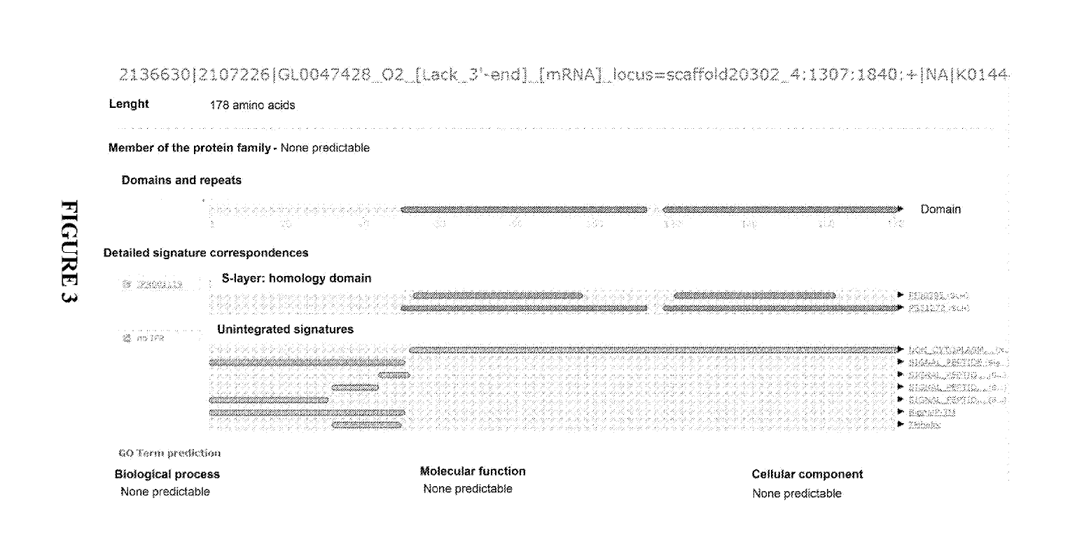

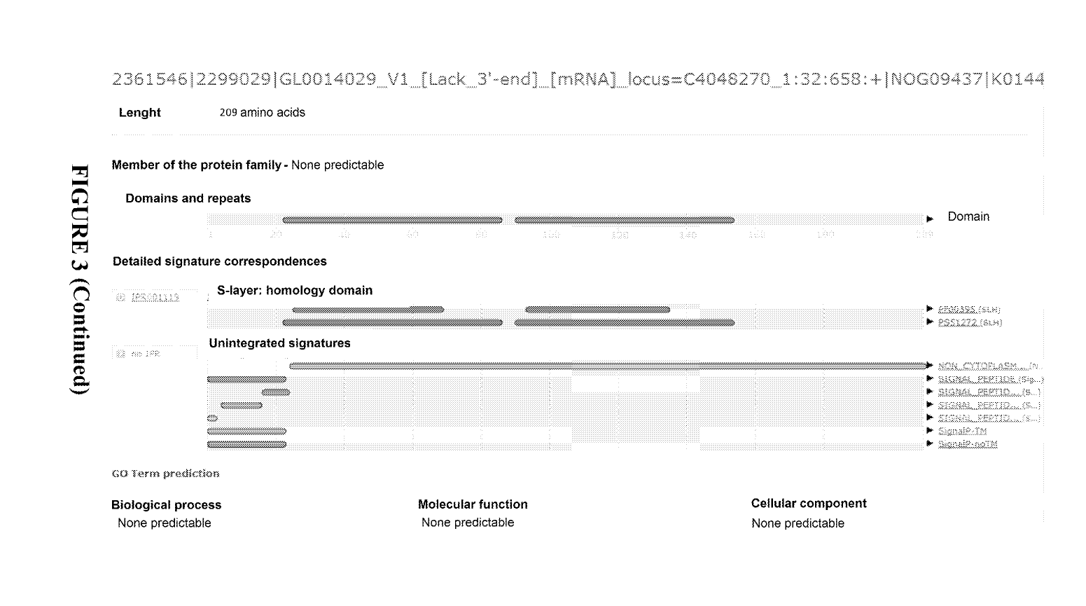

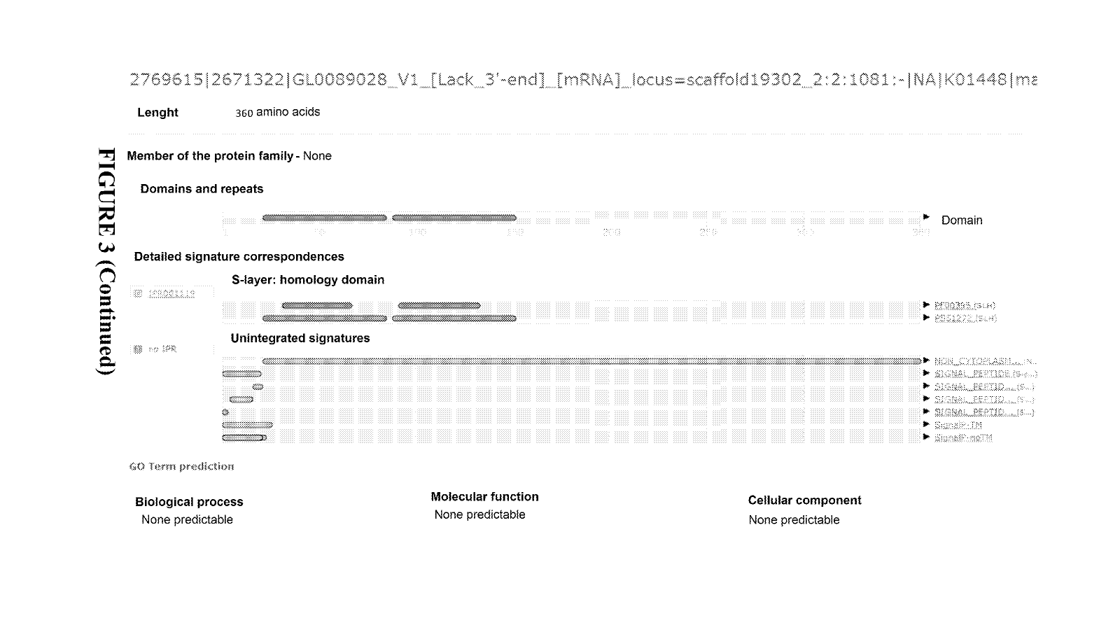

Three microbial proteins were discovered as being more abundant in the healthy subjects compared with the ill by a label-free shotgun tandem mass spectrometry method developed by the inventors (Guillot et al., 2013). Identification of the proteins rests on the mass matching technique and on querying the MetaHIT metagenomics database (Qin et al., 2010) using the bioinformatics tool X!TandemPipeline (http://pappso.inra.fr/bioinfo/xtandempipeline/). Their identifiers in the MetaHIT 2009 database reannotated in February 2014, their sequences, and the peptides seen in mass spectrometry and having allowed their identification appear in FIG. 1.

Materials and Methods

Twelve intestinal microbiotas (all intestinal microbial populations) are extracted from 12 fresh stool samples (w=2 g) collected from 6 Crohn's patients in remission and 6 controls with no intestinal pathology. The extraction procedure is that described by the inventors (Juste et al., 2014). It is based on harvesting bacterial cells at their floating density within a continuous Nycodenz gradient, pre-formed by freezing/thawing. The stool sample weighed down in Nycodenz is deposited beneath the preformed gradient and the bacterial cells return to their floating density during low-speed centrifugation (14,567×g, 45 min, 4° C.). The anaerobic nature and the integrity of the bacteria are thus preserved throughout the extraction process. The bacterial pellets, washed in 20 mM Tris, 138 mM NaCl, 2.7 mM KCl, 0.03% (w/v) Na-deoxycholate, pH 7.4, are kept at −80° C. A 1.5 ml volume of lysis buffer (8.75 M urea, 2.5 M thiourea, 5% (w/v) CHAPS, 75 mM DTT, and 31.2 mM spermine dihydrate base) is deposited on each still-frozen pellet. The chemical lysis is performed at room temperature for 1 h during which the samples are vortexed vigorously every 10 min. The lysates are centrifuged at very high speed (245,419×g, 1 h, 18° C.) and the supernatants are collected and brought to neutral pH with concentrated HCl. The proteins are purified with the PlusOne SDS-PAGE Kit (GE Healthcare), then assayed with the 2-D Quant Kit (GE Healthcare). All the samples are brought to a protein concentration of 4 μg/μl with Laemmli denaturing buffer.

For the discovery step, we use a label-free shotgun proteomics method with prefractionation of the sample on SDS-PAGE gel for better coverage of the proteome. Briefly, 60 μg of proteins from a Crohn's patient and 60 μg of proteins from a healthy subject migrate for about 45 min in an SDS-PAGE gel (4-12% NuPAGE Novex gel, 200V, 110 mA) mounted on an XCell SureLock™ device (Invitrogen). After migration, the gel is rinsed with water, stained with Coomassie blue and scanned. Each of the two lanes is cut into 20 equivalent bands and each of the 40 pieces of gel is subjected to reduction, alkylation and tryptic digestion according to a standard protocol (Shevchenko et al., 2007). Tryptic peptides are extracted from the gel with 50% acetonitrile, 0.2% formic acid, then dried under vacuum (SpeedVac), taken up in 25 μl of HPLC buffer (0.1% formic acid, 2% acetonitrile) and analyzed individually (i.e., 40 analyses in total) by coupled LC-MS/MS (NanoLC Ultra System, Eksigent) connected to a mass spectrometer (Q Exactive, Thermo). To that end, 4 μl of peptide extract is loaded on a precolumn (stationary phase: PepMap 100 C18, 5 μm; column: 300 μm ID, 5 mm, Dionex) with a 7.5 μl/min flow rate. After 3 min, the precolumn is connected to a separation column (stationary phase: C18 Biosphere, 3 μm; column: 75 μm ID, 150 μm, NanoSeparations) and the peptides are eluted by using a linear gradient (5-35%) of buffer B (80% acetonitrile, 0.1% formic acid) in buffer A (2% acetonitrile, 0.1% formic acid) for 40 min, for a total acquisition time of 50 min per piece of gel. Ionization is carried out via a ‘PicoTip’ needle emitter (20 μm ID, 360 μm OD, New Objective). The peptide ions are detected automatically in data-dependent mode with the following parameters: “full scan” MS (m/z 400-1400 Th) to measure the m/z ratio of the peptides and fragmentation of the peptides observed with a normalized collision energy set to 30% in HCD, under the control of the Xcalibur software (version 2.1, Thermo). In this study, only the doubly and triply charged precursor ions were selected to be fragmented in MS/MS with an exclusion window of 40 sec while having activated the auto-calibration mode on the ion 445.12003 (dimethylcyclosiloxane).

We use the X!Tandem software to compare the measured masses of the fragmented peptides with the theoretical fragmentation masses generated from the MetaHIT database [3,299,822 complete or incomplete ORFs (Qin et al., 2010) reannotated for their function and their taxonomy in February 2014], combined with the UniProtKB/Swiss-Prot H. sapiens database and our own contaminant database. The search parameters and the validation thresholds for the peptides and the proteins are those described recently by the inventors (Juste et al., 2014). To finish, we use the X!TandemPipeline software (http://pappso.inra.fr/bioinfo/xtandempipeline/) to summarize the results in the form of tables giving the number of fragmented peptide ions attributed to each protein, as well as the sequence of these peptides. By inference, this number makes it possible to estimate the quantity of said protein in several samples. In the present invention, and by this method called “spectral counting”, the total number of fragmentation spectra attributed to each protein was summed for the 20 fractions of the “Crohn” sample on the one hand, and the 20 fractions of the “Healthy” sample on the other hand. The sum of the spectra attributed to the proteins SEQ ID NO: 1, SEQ ID NO: 2 and SEQ ID NO: 3 is remarkably low in the “Crohn” sample compared with the “Healthy” sample.

To confirm these results, the 6 “Crohn” samples and the 6 “Healthy” samples prepared above (100 μg of purified protein from each sample) were analyzed by the targeted proteomics method called selected reaction monitoring (SRM) with labeling, detailed by the inventors (Juste et al., 2014). Tryptic peptides specific for (not shared with other proteins within the MetaHIT database) the proteins SEQ ID NO: 1, SEQ ID NO: 2 and SEQ ID NO: 3 are identified. Among them, the peptides already identified in label-free shotgun experiments and having fragmentation spectra of very good quality are singled out. The selected peptides have an ideal length of 7 to 25 amino acids and contain neither miscleavage nor methionine. The selection of transitions, the carrying out of microLC-SRM assays with labeling, the data analysis, the quality controls and the statistical analyses are those detailed by the inventors (Juste et al., 2014). In the present invention, and by this SRM method with labeling, the low abundance of the peptide sequences SEQ ID NO: 4 to SEQ ID NO: 13 and, by inference, the low abundance of the protein sequences SEQ ID NO: 1, SEQ ID NO: 2 and SEQ ID NO: 3, are confirmed among the ill subjects relative to the controls.

### REFERENCES

Artis D. Epithelial-cell recognition of commensal bacteria and maintenance of immune homeostasis in the gut. Nat Rev Immunol. 2008; 8:411-420
Beaugerie L, Brousse N, Bouvier A M, et al. Lymphoproliferative disorders in patients receiving thiopurines for inflammatory bowel disease: a prospective observational cohort study. Lancet. 2009; 374:1617-1625
Bird et al. [Science 242 (1988) 423]
Carbonnel F, Jantchou P, Monnet E, et al. Environmental risk factors in Crohn's disease and ulcerative colitis: an update. Gastroenterologie Clinique et Biologique. 2009; 33:S145-S157
Cho J H. The genetics and immunopathogenesis of inflammatory bowel disease. Nat Rev Immunol. 2008; 8:458
Cleynen I, Gonzalez J R, Figueroa C, et al. Genetic factors conferring an increased susceptibility to develop Crohn's disease also influence disease phenotype: results from the IBDchip European Project. Gut. 2013; 62:1556
Colombel J F, Sandborn W J, Reinisch W, et al. Infliximab, azathioprine, or combination therapy for Crohn's disease. N Engl J Med. 2010; 362:1383-1395
Gophna U, Sommerfeld K, Gophna S, et al. Differences between tissue-associated intestinal microfloras of patients with Crohn's disease and ulcerative colitis. J Clin Microbiol. 2006; 44:4136
Guillot A, Blon F, Valot B, et al. A strategy to go deep and fast into the collective proteome of gut microbes. EuPA Saint-Malo, 14-17 Oct. 2013
Harlow et al. Antibodies: A Laboratory Manual, CSH Press, 1988
Johnson G J, Cosnes J, Mansfield J C. Review article: smoking cessation as primary therapy to modify the course of Crohn's disease. Aliment Pharmacol Ther. 2005; 21:921-931
Juillerat P, Pittet V, Mottet C, et al. Appropriateness of early management of newly diagnosed Crohn's disease in a European population-based cohort. Scand J Gastroenterol. 2010; 45:1449-1456
Juste C, Kreil D P, Beauvallet C, et al. Bacterial protein signals are associated with Crohn's disease. Gut. 2014; DOI:10.1136/gutjnl-2012-303786
Kleessen B, Kroesen A J, Buhr H J, et al. Mucosal and invading bacteria in patients with inflammatory bowel diseasecompared with controls. Scand J Gastroenterol. 2002; 37:1034
Landers C J, Cohavy O, Misra R, et al. Selected loss of tolerance evidenced by Crohn's disease-associated immune responses to auto- and microbial antigens. Gastroenterology. 2002; 123:689-6993
Laroux F S, Pavlick K P, Wolf R E, et al. Dysregulation of intestinal mucosal immunity: implications in inflammatory bowel disease. News Physiol Sci 2001; 16:272-277
Mangin I, Bonnet R, Seksik P, et al. Molecular inventory of faecal microflora in patients with Crohn's disease. FEMS Microbiol Ecol. 2004; 50:25
Manichanh C, Rigottier-Gois L, Bonnaud E, et al. Reduced diversity of faecal microbiota in Crohn's disease revealed by a metagenomic approach. Gut. 2006; 55:205
Mathew C G. New links to the pathogenesis of Crohn disease provided by genome-wide association scans. Nat Rev Genet 2008; 9:9-14
Peyrin-Biroulet L, Loftus E V, Jr., Colombel J F, et al. The natural history of adult Crohn's disease in population-based cohorts. Am J Gastroenterol. 2010; 105:289-297
Peyrin-Biroulet L, Khosrotehrani K, Carrat F, et al. Increased risk for nonmelanoma skin cancers in patients who receive thiopurines for inflammatory bowel disease. Gastroenterology. 2011; 141:1621-1628 e1625
Qin J, Ruiqiang L, Raes J, et al. A human gut microbial gene catalog established by deep metagenomic sequencing. Nature 2010; 464:59-65
Sambrook, Fritsch, Maniatis (1989) Molecular Cloning, Cold Spring Harbor Laboratory Press
Scanlan P D, Shanahan F, O'Mahony C, et al. Culture-independent analyses of temporal variation of the dominant fecal microbiota and targeted bacterial subgroups in Crohn's disease. J Clin Microbiol. 2006; 44:3980
Seksik P, Rigottier-Gois L, Gramet G, et al. Alterations of the dominant faecal bacterial groups in patients with Crohn's disease of the colon. Gut. 2003; 52:237
Sokol H, Beaugerie L. Inflammatory bowel disease and lymphoproliferative disorders: the dust is starting to settle. Gut. 2009; 58:1427-1436
Shevchenko A, Tomas H, Havlis J, et al. In-gel digestion for mass spectrometric characterization of proteins and proteomes. Nat Protoc 2007, 1: 2856-60.
Ward et al. Nature 341 (1989) 544

1-8. (canceled)

9. A method for detecting the presence of a bacterium in a subject suspected of having Crohn's disease comprising detecting the expression of a peptide sequence comprising an amino acid sequence selected from SEQ ID NO: 2, SEQ ID NO: 1, SEQ ID NO: 3 and a 7 to 25 amino acid fragment thereof, or comprising a nucleic acid encoding said peptide sequence by a bacterium, or a portion of said bacterium in a sample comprising fecal matter, fecal water and/or intestinal epithelial tissue from a subject to be tested for Crohn's disease.

10. The method according to claim 9, characterized in that the fragment of the amino acid sequence comprises SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12 or SEQ ID NO: 13.

11. The method according to claim 9, characterized in that the detection of said at least one bacterium or a portion thereof is carried out by targeted quantitative proteomics.

12. The method according to claim 9, characterized in that it comprises:
contacting, in vitro or ex vivo, a sample comprising fecal matter, fecal water and/or intestinal epithelial tissue from the subject to be tested with a compound binding all or a portion of said at least one bacterium; and
detecting the bond between said compound and all or a portion of said at least one bacterium when it is present in the sample.

13. The method according to claim 9, characterized in that the subject is an animal.

14. The method according to claim 13, wherein said animal is a human.

15. The method according to claim 14, wherein said human has a chronic digestive disorder, an unclassified chronic inflammatory bowel disease (IBD), or a family history of IBD.

16. A method for obtaining information useful in the diagnosis of Crohn's disease comprising implementing, in vitro or ex vivo, a detection method according to claim 9 on a sample comprising fecal matter, fecal water and/or intestinal epithelial tissue from a subject to be tested, characterized in that the absence of the bacterium or a portion thereof, or the detection of the bacteria or portions thereof, in a quantity below a control value within the sample, makes it possible to diagnose Crohn's disease in said subject, the presence within the sample of the bacteria or portions thereof in a quantity above said control value making it possible, conversely, to exclude the existence of Crohn's disease in said subject.

17. A method for evaluating, in a subject, the therapeutic efficacy of a treatment for Crohn's disease, said method comprising implementing, in vitro or ex vivo, a detection method according to claim 9 on a sample comprising fecal matter, fecal water and/or intestinal epithelial tissue from a subject treated for Crohn's disease, the appearance of or an increase in the quantity of bacteria or portions thereof revealing the efficacy of said treatment, and the disappearance of or the reduction in the quantity of bacteria or portions thereof revealing, conversely, the inefficacy of said treatment, when compared with a control value, for example when compared with the quantity of bacteria or portions thereof detected in said subject before said subject was treated for Crohn's disease.
